SEQUENCE LISTING (71 sequences)

SEQ ID NO: 1 - RNA, 25 nt, Artificial Sequence
  oligo
uagacuuccacagaacucuucaaag

SEQ ID NO: 2 - RNA, 25 nt, Artificial Sequence
  oligo
  modified_base (1)..(1): cm
  modified_base (2)..(2): um
  modified_base (3)..(3): um
  modified_base (4)..(4): um
  modified_base (5)..(5): gm
  misc_feature (6)..(6): 2′ Me
  misc_feature (7)..(7): 2′ Me
  modified_base (8)..(8): gm
  misc_feature (9)..(9): 2′ Me
  modified_base (10)..(10): gm
  modified_base (11)..(11): um
  modified_base (12)..(12): um
  modified_base (16)..(16): um
  modified_base (17)..(17): gm
  modified_base (18)..(18): gm
  misc_feature (19)..(19): 2′ Me
  misc_feature (20)..(20): 2′ Me
  modified_base (21)..(21): gm
  modified_base (22)..(22): um
  modified_base (23)..(23): cm
  modified_base (24)..(24): um
  misc_feature (25)..(25): 2′ Me
cuuugaagaguucuguggaagucua

SEQ ID NO: 3 - RNA, 25 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  misc_feature (20)..(20): 2′ F
  modified_base (21)..(21): cm
  misc_feature (22)..(22): 2′ F
  misc_feature (23)..(23): 2′ Me
  misc_feature (24)..(24): 2′ F
  modified_base (25)..(25): gm
uagacuuccacagaacucuucaaag

SEQ ID NO: 4 - RNA, 25 nt, Artificial Sequence
  oligo
  misc_feature (1)..(1): 2′ F
  modified_base (2)..(2): um
  misc_feature (3)..(3): 2′ F
  modified_base (4)..(4): um
  misc_feature (5)..(5): 2′ F
  misc_feature (6)..(6): 2′ Me
  misc_feature (7)..(7): 2′ F
  modified_base (8)..(8): gm
  misc_feature (9)..(9): 2′ F
  modified_base (10)..(10): gm
  misc_feature (11)..(11): 2′ F
  modified_base (12)..(12): um
  misc_feature (13)..(13): 2′ F
  misc_feature (14)..(14): 2′ F
  misc_feature (15)..(15): 2′ F
  modified_base (16)..(16): um
  misc_feature (17)..(17): 2′ F
  modified_base (18)..(18): gm
  misc_feature (19)..(19): 2′ F
  misc_feature (20)..(20): 2′ Me
  misc_feature (21)..(21): 2′ F
  modified_base (22)..(22): um
  misc_feature (23)..(23): 2′ F
  modified_base (24)..(24): um
  misc_feature (25)..(25): 2′ F
cuuugaagaguucuguggaagucua

SEQ ID NO: 5 - RNA, 19 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
uagacuuccacagaacucu

SEQ ID NO: 6 - RNA, 19 nt, Artificial Sequence
  oligo
  misc_feature (1)..(1): 2′ F
  modified_base (2)..(2): gm
  misc_feature (3)..(3): 2′ F
  modified_base (4)..(4): gm
  misc_feature (5)..(5): 2′ F
  modified_base (6)..(6): um
  misc_feature (7)..(7): 2′ F
  misc_feature (8)..(8): 2′ F
  misc_feature (9)..(9): 2′ F
  modified_base (10)..(10): um
  misc_feature (11)..(11): 2′ F
  modified_base (12)..(12): gm
  misc_feature (13)..(13): 2′ F
  misc_feature (14)..(14): 2′ Me
  misc_feature (15)..(15): 2′ F
  modified_base (16)..(16): um
  misc_feature (17)..(17): 2′ F
  modified_base (18)..(18): um
  misc_feature (19)..(19): 2′ F
agaguucuguggaagucua

SEQ ID NO: 7 - RNA, 19 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(12): flip in NC
  misc_feature (8)..(8): 2′ F
  modified_base (9)..(9): gm
  misc_feature (10)..(10): 2′ F
  modified_base (11)..(11): gm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
uagacuuggugugaacucu

SEQ ID NO: 8 - RNA, 19 nt, Artificial Sequence
  oligo
  misc_feature (1)..(1): 2′ F
  modified_base (2)..(2): gm
  misc_feature (3)..(3): 2′ F
  modified_base (4)..(4): gm
  misc_feature (5)..(5): 2′ F
  modified_base (6)..(6): um
  misc_feature (7)..(7): 2′ F
  misc_feature (8)..(12): flip in NC
  misc_feature (8)..(8): 2′ F
  misc_feature (9)..(9): 2′ F
  misc_feature (10)..(10): 2′ Me
  misc_feature (11)..(11): 2′ F
  modified_base (12)..(12): cm
  misc_feature (13)..(13): 2′ F
  misc_feature (14)..(14): 2′ Me
  misc_feature (15)..(15): 2′ F
  modified_base (16)..(16): um
  misc_feature (17)..(17): 2′ F
  modified_base (18)..(18): um
  misc_feature (19)..(19): 2′ F
agaguucacaccaagucua

SEQ ID NO: 9 - RNA, 33 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  modified_base (20)..(20): um
  misc_feature (21)..(21): 2′ F
  misc_feature (22)..(22): 2′ F
  misc_feature (23)..(23): 2′ F
  modified_base (24)..(24): um
  misc_feature (25)..(25): 2′ F
  modified_base (26)..(26): gm
  misc_feature (27)..(27): 2′ F
  misc_feature (28)..(28): 2′ Me
  misc_feature (29)..(29): 2′ F
  modified_base (30)..(30): um
  misc_feature (31)..(31): 2′ F
  modified_base (32)..(32): um
  misc_feature (33)..(33): 2′ F
uagacuuccacagaacucuucuguggaagucua

SEQ ID NO: 10 - RNA, 32 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  misc_feature (20)..(20): 2′ F
  misc_feature (21)..(21): 2′ F
  misc_feature (22)..(22): 2′ F
  modified_base (23)..(23): um
  misc_feature (24)..(24): 2′ F
  modified_base (25)..(25): gm
  misc_feature (26)..(26): 2′ F
  misc_feature (27)..(27): 2′ Me
  misc_feature (28)..(28): 2′ F
  modified_base (29)..(29): um
  misc_feature (30)..(30): 2′ F
  modified_base (31)..(31): um
  misc_feature (32)..(32): 2′ F
uagacuuccacagaacucucuguggaagucua

SEQ ID NO: 11 - RNA, 31 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (20)..(20): 2′ F
  misc_feature (21)..(21): 2′ F
  modified_base (22)..(22): um
  misc_feature (23)..(23): 2′ F
  modified_base (24)..(24): gm
  misc_feature (25)..(25): 2′ F
  misc_feature (26)..(26): 2′ Me
  misc_feature (27)..(27): 2′ F
  modified_base (28)..(28): um
  misc_feature (29)..(29): 2′ F
  modified_base (30)..(30): um
  misc_feature (31)..(31): 2′ F
uagacuuccacagaacucuuguggaagucua

SEQ ID NO: 12 - RNA, 30 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (20)..(20): 2′ F
  modified_base (21)..(21): um
  misc_feature (22)..(22): 2′ F
  modified_base (23)..(23): gm
  misc_feature (24)..(24): 2′ F
  misc_feature (25)..(25): 2′ Me
  misc_feature (26)..(26): 2′ F
  modified_base (27)..(27): um
  misc_feature (28)..(28): 2′ F
  modified_base (29)..(29): um
  misc_feature (30)..(30): 2′ F
uagacuuccacagaacucuguggaagucua

SEQ ID NO: 13 - RNA, 29 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  misc_feature (17)..(17): 2′ F
  misc_feature (18)..(18): 2′ F
  misc_feature (19)..(19): 2′ F
  modified_base (20)..(20): um
  misc_feature (21)..(21): 2′ F
  modified_base (22)..(22): gm
  misc_feature (23)..(23): 2′ F
  misc_feature (24)..(24): 2′ Me
  misc_feature (25)..(25): 2′ F
  modified_base (26)..(26): um
  misc_feature (27)..(27): 2′ F
  modified_base (28)..(28): um
  misc_feature (29)..(29): 2′ F
uagacuuccacagaacucuuggaagucua

SEQ ID NO: 14 - RNA, 33 nt, Artificial Sequence; identical to SEQ ID NO: 9
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (14)..(20): Phosphorothioate linkage
  misc_feature (15)..(15): 2′ Me
  misc_feature (16)..(16): 2′ F
  modified_base (17)..(17): um
  misc_feature (18)..(18): 2′ F
  modified_base (19)..(19): um
  misc_feature (21)..(21): 2′ F
  misc_feature (22)..(22): 2′ F
  misc_feature (23)..(23): 2′ F
  modified_base (24)..(24): um
  misc_feature (25)..(25): 2′ F
  modified_base (26)..(26): gm
  misc_feature (27)..(27): 2′ F
  misc_feature (28)..(28): 2′ Me
  misc_feature (29)..(29): 2′ F
  modified_base (30)..(30): um
  misc_feature (31)..(31): 2′ F
  modified_base (32)..(32): um
  misc_feature (33)..(33): 2′ F

SEQ ID NO: 15 - RNA, 32 nt, Artificial Sequence; identical to SEQ ID NO: 10
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(19): Phosphorothioate linkage
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (20)..(20): 2′ F
  misc_feature (21)..(21): 2′ F
  misc_feature (22)..(22): 2′ F
  modified_base (23)..(23): um
  misc_feature (24)..(24): 2′ F
  modified_base (25)..(25): gm
  misc_feature (26)..(26): 2′ F
  misc_feature (27)..(27): 2′ Me
  misc_feature (28)..(28): 2′ F
  modified_base (29)..(29): um
  misc_feature (30)..(30): 2′ F
  modified_base (31)..(31): um
  misc_feature (32)..(32): 2′ F

SEQ ID NO: 16 - RNA, 31 nt, Artificial Sequence; identical to SEQ ID NO: 11
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(20): Phosphorothioate linkage
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (21)..(21): 2′ F
  modified_base (22)..(22): um
  misc_feature (23)..(23): 2′ F
  modified_base (24)..(24): gm
  misc_feature (25)..(25): 2′ F
  misc_feature (26)..(26): 2′ Me
  misc_feature (27)..(27): 2′ F
  modified_base (28)..(28): um
  misc_feature (29)..(29): 2′F
  modified_base (30)..(30): um
  misc_feature (31)..(31): 2′ F

SEQ ID NO: 17 - RNA, 30 nt, Artificial Sequence; identical to SEQ ID NO: 12
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (13)..(19): Phosphorothioate linkage
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (20)..(20): 2′ F
  modified_base (21)..(21): um
  misc_feature (22)..(22): 2′ F
  modified_base (23)..(23): gm
  misc_feature (24)..(24): 2′ F
  misc_feature (25)..(15): 2′ Me
  misc_feature (26)..(26): 2′ F
  modified_base (27)..(27): um
  misc_feature (28)..(28): 2′ F
  modified_base (29)..(29): um
  misc_feature (30)..(30): 2′ F

SEQ ID NO: 18 - RNA, 29 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  misc_feature (10)..(20): Phosphorothioate linkage
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  misc_feature (17)..(17): 2′ F
  misc_feature (18)..(18): 2′ F
  misc_feature (19)..(19): 2′ F
  misc_feature (21)..(21): 2′ F
  modified_base (22)..(22): gm
  misc_feature (23)..(23): 2′ F
  misc_feature (24)..(24): 2′ Me
  misc_feature (25)..(25): 2′ F
  modified_base (26)..(26): um
  misc_feature (27)..(27): 2′ F
  modified_base (28)..(28): um
  misc_feature (29)..(29): 2′ F
uagacuuccacagaacucuuggaagucua

SEQ ID NO: 19 - RNA, 25 nt, Artificial Sequence
  oligo
  modified_base (1)..(1): cm
  modified_base (2)..(2): um
  modified_base (3)..(3): um
  modified_base (4)..(4): um
  modified_base (5)..(5): gm
  misc_feature (6)..(6): 2′ Me
  misc_feature (7)..(7): 2′ Me
  modified_base (8)..(8): gm
  misc_feature (9)..(9): 2′ Me
  modified_base (10)..(10): gm
  modified_base (11)..(11): um
  modified_base (12)..(12): um
  modified_base (16)..(16): um
  modified_base (17)..(17): gm
  modified_base (18)..(18): gm
  misc_feature (19)..(19): 2′ Me
  misc_feature (20)..(20): 2′ Me
  modified_base (21)..(21): gm
  modified_base (22)..(22): um
  modified_base (23)..(23): cm
  modified_base (24)..(24): um
  misc_feature (25)..(25): 2′ Me
  misc_feature (25)..(25): GN linker-2xGalNAc
cuuugaagaguucuguggaagucua

SEQ ID NO: 20 - RNA, 25 nt, Artificial Sequence
  oligo
  misc_feature (1)..(1): 2′ F
  modified_base (2)..(2): um
  misc_feature (3)..(3): 2′ F
  modified_base (4)..(4): um
  misc_feature (5)..(5): 2′ F
  misc_feature (6)..(6): 2′ Me
  misc_feature (7)..(7): 2′ F
  modified_base (8)..(8): gm
  misc_feature (9)..(9): 2′ F
  modified_base (10)..(10): gm
  misc_feature (11)..(11): 2′ F
  modified_base (12)..(12): um
  misc_feature (13)..(13): 2′ F
  misc_feature (14)..(14): 2′ F
  misc_feature (15)..(15): 2′ F
  modified_base (16)..(16): um
  misc_feature (17)..(17): 2′ F
  modified_base (18)..(18): gm
  misc_feature (19)..(19): 2′ F
  misc_feature (20)..(20): 2′ Me
  misc_feature (21)..(21): 2′ F
  modified_base (22)..(22): um
  misc_feature (23)..(23): 2′ F
  modified_base (24)..(24): um
  misc_feature (25)..(25): GN linker-2xGalNAc
  misc_feature (25)..(25): 2′ F
cuuugaagaguucuguggaagucua

SEQ ID NO: 21 - RNA, 19 nt, Artificial Sequence
  oligo
  misc_feature (1)..(1): 2′ F
  modified_base (2)..(2): gm
  misc_feature (3)..(3): 2′ F
  modified_base (4)..(4): gm
  misc_feature (5)..(5): 2′ F
  modified_base (6)..(6): um
  misc_feature (7)..(7): 2′ F
  misc_feature (8)..(8): 2′ F
  misc_feature (9)..(9): 2′ F
  modified_base (10)..(10): um
  misc_feature (11)..(11): 2′ F
  modified_base (12)..(12): gm
  misc_feature (13)..(13): 2′ F
  misc_feature (14)..(14): 2′ Me
  misc_feature (15)..(15): 2′ F
  modified_base (16)..(16): um
  misc_feature (17)..(17): 2′ F
  modified_base (18)..(18): um
  misc_feature (19)..(19): 2′ F
  misc_feature (19)..(19): GN linker-2xGalNAc
agaguucuguggaagucua

SEQ ID NO: 22 - RNA, 19 nt, Artificial Sequence
  oligo
  misc_feature (1)..(1): 2′ F
  modified_base (2)..(2): gm
  misc_feature (3)..(3): 2′ F
  modified_base (4)..(4): gm
  misc_feature (5)..(5): 2′ F
  modified_base (6)..(6): um
  misc_feature (7)..(7): 2′ F
  misc_feature (8)..(8): 2′ F
  misc_feature (8)..(12): flip in NC
  misc_feature (9)..(9): 2′ F
  misc_feature (10)..(10): 2′ Me
  misc_feature (11)..(11): 2′ F
  modified_base (12)..(12): cm
  misc_feature (13)..(13): 2′ F
  misc_feature (14)..(14): 2′ Me
  misc_feature (15)..(15): 2′ F
  modified_base (16)..(16): um
  misc_feature (17)..(17): 2′ F
  modified_base (18)..(18): um
  misc_feature (19)..(19): 2′ F
  misc_feature (19)..(19): GN linker-2xGalNAc
agaguucacaccaagucua

SEQ ID NO: 23 - RNA, 33 nt, Artificial Sequence; identical to SEQ ID NO: 9
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  modified_base (20)..(20): um
  misc_feature (21)..(21): 2′ F
  misc_feature (22)..(22): 2′ F
  misc_feature (23)..(23): 2′ F
  modified_base (24)..(24): um
  misc_feature (25)..(25): 2′ F
  modified_base (26)..(26): gm
  misc_feature (27)..(27): 2′ F
  misc_feature (28)..(28): 2′ ME
  misc_feature (29)..(29): 2′ F
  modified_base (30)..(30): um
  misc_feature (31)..(31): 2′ F
  modified_base (32)..(32): um
  misc_feature (33)..(33): 2′ F
  misc_feature (33)..(33): GN linker-2xGalNAc

SEQ ID NO: 24 - RNA, 32 nt, Artificial Sequence; identical to SEQ ID NO: 10
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  misc_feature (20)..(20): 2′ F
  misc_feature (21)..(21): 2′ F
  misc_feature (22)..(22): 2′ F
  modified_base (23)..(23): um
  misc_feature (24)..(24): 2′ F
  modified_base (25)..(25): gm
  misc_feature (26)..(26): 2′ F
  misc_feature (27)..(27): 2′ Me
  misc_feature (28)..(28): 2′ F
  modified_base (29)..(29): um
  misc_feature (30)..(30): 2′ F
  modified_base (31)..(31): um
  misc_feature (32)..(32): 2′ F
  misc_feature (32)..(32): GN linker-2xGalNAc

SEQ ID NO: 25 - RNA, 31 nt, Artificial Sequence; identical to SEQ ID NO: 11
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (20)..(20): 2′ F
  misc_feature (21)..(21): 2′ F
  modified_base (22)..(22): um
  misc_feature (23)..(23): 2′ F
  modified_base (24)..(24): gm
  misc_feature (25)..(25): 2′ F
  misc_feature (26)..(26): 2′ Me
  misc_feature (27)..(27): 2′ F
  modified_base (28)..(28): um
  misc_feature (29)..(29): 2′ F
  modified_base (30)..(30): um
  misc_feature (31)..(31): 2′ F
  misc_feature (31)..(31): GN linker-2xGalNAc

SEQ ID NO: 26 - RNA, 30 nt, Artificial Sequence; identical to SEQ ID NO: 12
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (20)..(20): 2′ F
  modified_base (21)..(21): um
  misc_feature (22)..(22): 2′ F
  modified_base (23)..(23): gm
  misc_feature (24)..(24): 2′ F
  misc_feature (25)..(25): 2′ Me
  misc_feature (26)..(26): 2′ F
  modified_base (27)..(27): um
  misc_feature (28)..(28): 2′ F
  modified_base (29)..(29): um
  misc_feature (30)..(30): 2′ F
  misc_feature (30)..(30): GN linker-2xGalNAc

SEQ ID NO: 27 - RNA, 29 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  misc_feature (17)..(17): 2′ F
  misc_feature (18)..(18): 2′ F
  misc_feature (19)..(19): 2′ F
  modified_base (20)..(20): um
  misc_feature (21)..(21): 2′ F
  modified_base (22)..(22): gm
  misc_feature (23)..(23): 2′ F
  misc_feature (24)..(24): 2′ Me
  misc_feature (25)..(25): 2′ F
  modified_base (26)..(26): um
  misc_feature (27)..(27): 2′ F
  modified_base (28)..(28): um
  misc_feature (29)..(29): 2′ F
  misc_feature (29)..(29): GN linker-2xGalNAc
uagacuuccacagaacucuuggaagucua

SEQ ID NO: 28 - RNA, 33 nt, Artificial Sequence; identical to SEQ ID NO: 9
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  modified_base (19)..(19): um
  misc_feature (21)..(21): 2′ F
  misc_feature (22)..(22): 2′ F
  misc_feature (23)..(23): 2′ F
  modified_base (24)..(24): um
  misc_feature (25)..(25): 2′ F
  modified_base (26)..(26): gm
  misc_feature (27)..(27): 2′ F
  misc_feature (28)..(28): 2′ Me
  misc_feature (29)..(29): 2′ F
  modified_base (30)..(30): um
  misc_feature (31)..(31): 2′ F
  modified_base (32)..(32): um
  misc_feature (33)..(33): 2′ F
  misc_feature (33)..(33): GN linker-2xGalNAc

SEQ ID NO: 29 - RNA, 32 nt, Artificial Sequence; identical to SEQ ID NO: 10
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (14)..(14): 2′ F
  misc_feature (14)..(19): Phosphorothioate linkage
  misc_feature (15)..(15): 2′ Me
  misc_feature (16)..(16): 2′ F
  modified_base (17)..(17): um
  misc_feature (18)..(18): 2′ F
  misc_feature (20)..(20): 2′ F
  misc_feature (21)..(21): 2′ F
  misc_feature (22)..(22): 2′ F
  modified_base (23)..(23): um
  misc_feature (24)..(24): 2′ F
  modified_base (25)..(25): gm
  misc_feature (26)..(26): 2′ F
  misc_feature (27)..(27): 2′ Me
  misc_feature (28)..(28): 2′ F
  modified_base (29)..(29): um
  misc_feature (30)..(30): 2′ F
  modified_base (31)..(31): um
  misc_feature (32)..(32): 2′ F
  misc_feature (32)..(32): GN linker-2xGalNAc

SEQ ID NO: 30 - RNA, 31 nt, Artificial Sequence; identical to SEQ ID NO: 11
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  misc_feature (12)..(20): Phosphorothioate linkage
  modified_base (13)..(13): gm
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (19)..(19): 2′ F
  misc_feature (21)..(21): 2′ F
  modified_base (22)..(22): um
  misc_feature (23)..(23): 2′ F
  modified_base (24)..(24): gm
  misc_feature (25)..(25): 2′ F
  misc_feature (26)..(26): 2′ Me
  misc_feature (27)..(27): 2′ F
  modified_base (28)..(28): um
  misc_feature (29)..(29): 2′ F
  modified_base (30)..(30): um
  misc_feature (31)..(31): 2′ F
  misc_feature (31)..(31): GN linker-2xGalNAc

SEQ ID NO: 31 - RNA, 30 nt, Artificial Sequence; identical to SEQ ID NO: 12
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  modified_base (11)..(11): cm
  misc_feature (12)..(12): 2′ F
  modified_base (13)..(13): gm
  misc_feature (13)..(19): Phosphorothioate linkage
  misc_feature (14)..(14): 2′ F
  misc_feature (15)..(15): 2′ Me
  misc_feature (16)..(16): 2′ F
  modified_base (17)..(17): um
  misc_feature (18)..(18): 2′ F
  misc_feature (20)..(20): 2′ F
  modified_base (21)..(21): um
  misc_feature (22)..(22): 2′ F
  modified_base (23)..(23): gm
  misc_feature (24)..(24): 2′ F
  misc_feature (25)..(25): 2′ Me
  misc_feature (26)..(26): 2′ F
  modified_base (27)..(27): um
  misc_feature (28)..(28): 2′ F
  modified_base (29)..(29): um
  misc_feature (30)..(30): 2′ F
  misc_feature (30)..(30): GN linker-2xGalNAc

SEQ ID NO: 32 - RNA, 29 nt, Artificial Sequence
  oligo
  modified_base (1)..(1): um
  misc_feature (2)..(2): 2′ F
  modified_base (3)..(3): gm
  misc_feature (4)..(4): 2′ F
  modified_base (5)..(5): cm
  misc_feature (6)..(6): 2′ F
  modified_base (7)..(7): um
  misc_feature (8)..(8): 2′ F
  modified_base (9)..(9): cm
  misc_feature (10)..(10): 2′ F
  misc_feature (10)..(20): Phosphorothioate linker
  modified_base (11)..(11): cm
  misc_feature (12)..(12): 2′ F
  modified_base (13)..(13): gm
  misc_feature (14)..(14): 2′ F
  misc_feature (15)..(15): 2′ Me
  misc_feature (16)..(16): 2′ F
  misc_feature (17)..(17): 2′ F
  misc_feature (18)..(18): 2′ F
  misc_feature (19)..(19): 2′ F
  misc_feature (21)..(21): 2′ F
  modified_base (22)..(22): gm
  misc_feature (23)..(23): 2′ F
  misc_feature (24)..(24): 2′ Me
  misc_feature (25)..(25): 2′ F
  modified_base (26)..(26): um
  misc_feature (27)..(27): 2′ F
  modified_base (28)..(28): um
  misc_feature (29)..(29): 2′ F
  misc_feature (29)..(29): GN linker - 3xGalNac
uagacuuccacagaacucuuggaagucua

SEQ ID NO: 33 - RNA, 23 nt, Artificial Sequence
  oligo
  modified_base (1)..(1): um
  misc_feature (2)..(2): 2′ F
  misc_feature (3)..(3): 2′ Me
  misc_feature (4)..(4): 2′ F
  misc_feature (5)..(5): 2′ Me
  misc_feature (6)..(6): 2′ F
  misc_feature (7)..(7): 2′ Me
  misc_feature (8)..(8): 2′ F
  modified_base (9)..(9): cm
  misc_feature (10)..(10): 2′ F
  misc_feature (11)..(11): 2′ Me
  misc_feature (12)..(12): 2′ F
  misc_feature (13)..(13): 2′ Me
  misc_feature (14)..(14): 2′ F
  modified_base (15)..(15): cm
  misc_feature (16)..(16): 2′ F
  modified_base (17)..(17): cm
  misc_feature (18)..(18): 2′ F
  modified_base (19)..(19): gm
  misc_feature (20)..(20): 2′ F
  modified_base (21)..(21): um
  misc_feature (21)..(23): Phosphorothioate linker
  misc_feature (22)..(22): 2′ F
  modified_base (23)..(23): um
uuauagagcaagaacacuguuuu

SEQ ID NO: 34 - RNA, 21 nt, Artificial Sequence
  oligo
  misc_feature (1)..(1): 2′ F
  misc_feature (2)..(2): 2′ Me
  misc_feature (3)..(3): 2′ F
  misc_feature (4)..(4): 2′ Me
  misc_feature (5)..(5): 2′ F
  modified_base (6)..(6): um
  misc_feature (7)..(7): 2′ F
  modified_base (8)..(8): um
  misc_feature (9)..(9): 2′ F
  modified_base (10)..(10): cm
  misc_feature (11)..(11): 2′ F
  modified_base (12)..(12): um
  misc_feature (13)..(13): 2′ F
  modified_base (14)..(14): cm
  misc_feature (15)..(15): 2′ F
  modified_base (16)..(16): cm
  misc_feature (17)..(17): 2′ F
  misc_feature (18)..(18): 2′ Me
  misc_feature (19)..(19): 2′ F
  misc_feature (20)..(20): 2′ Me
  misc_feature (21)..(21): 2′ F
  misc_feature (21)..(21): GN linker - 3xGalNac
aacaguguucuugcucuauaa

SEQ ID NO: 35 - RNA, 19 nt, Artificial Sequence
  oligo
  misc_feature (1)..(1): 2′ Me
  misc_feature (1)..(3): Phosphorothioate linker
  misc_feature (2)..(2): 2′ F
  modified_base (3)..(3): cm
  misc_feature (4)..(4): 2′ F
  misc_feature (5)..(5): 2′ Me
  misc_feature (6)..(6): 2′ F
  misc_feature (7)..(7): 2′ Me
  misc_feature (8)..(8): 2′ F
  modified_base (9)..(9): gm
  misc_feature (10)..(10): 2′ F
  misc_feature (11)..(11): 2′ Me
  misc_feature (12)..(12): 2′ F
  modified_base (13)..(13): cm
  misc_feature (14)..(14): 2′ F
  modified_base (15)..(15): gm
  misc_feature (16)..(16): 2′ F
  modified_base (17)..(17): um
  misc_feature (17)..(19): Phosphorothioate linker
  misc_feature (18)..(18): 2′ F
  misc_feature (19)..(19): 2′ Me
aaccagaagaagcagguga

SEQ ID NO: 36 - RNA, 19 nt, Artificial Sequence
  oligo
  misc_feature (1)..(1): 2′ F
  misc_feature (1)..(1): GN linker - 3xGalNac
  modified_base (2)..(2): cm
  misc_feature (3)..(3): 2′ F
  modified_base (4)..(4): cm
  misc_feature (5)..(5): 2′ F
  modified_base (6)..(6): um
  misc_feature (7)..(7): 2′ F
  modified_base (8)..(8): cm
  misc_feature (9)..(9): 2′ F
  modified_base (10)..(10): um
  misc_feature (11)..(11): 2′ F
  modified_base (12)..(12): um
  misc_feature (13)..(13): 2′ F
  modified_base (14)..(14): cm
  misc_feature (15)..(15): 2′ F
  modified_base (16)..(16): gm
  misc_feature (17)..(17): 2′ F
  misc_feature (17)..(19): Phosphorothioate linker
  modified_base (18)..(18): um
  misc_feature (19)..(19): 2′ F
ucaccugcuucuucugguu

SEQ ID NO: 37 - RNA, 25 nt, Artificial Sequence
  unmodified oligo 2
cuuugaagaguucuguggaagucua

SEQ ID NO: 38 - RNA, 25 nt, Artificial Sequence
  unmodified oligo 3
uagacuuccacagaacucuucaaag

SEQ ID NO: 39 - RNA, 25 nt, Artificial Sequence
  unmodified oligo 4
cuuugaagaguucuguggaagucua

SEQ ID NO: 40 - RNA, 19 nt, Artificial Sequence
  unmodified oligo 5
uagacuuccacagaacucu

SEQ ID NO: 41 - RNA, 19 nt, Artificial Sequence
  unmodified oligo 6
agaguucuguggaagucua

SEQ ID NO: 42 - RNA, 19 nt, Artificial Sequence
  unmodified oligo 7
uagacuuggugugaacucu

SEQ ID NO: 43 - RNA, 19 nt, Artificial Sequence
  unmodified oligo 8
agaguucacaccaagucua

SEQ ID NO: 44 - RNA, 33 nt, Artificial Sequence; identical to SEQ ID NO: 9
  unmodified oligo 9

SEQ ID NO: 45 - RNA, 32 nt, Artificial Sequence; identical to SEQ ID NO: 10
  unmodified oligo 10

SEQ ID NO: 46 - RNA, 31 nt, Artificial Sequence; identical to SEQ ID NO: 11
  unmodified oligo 11

SEQ ID NO: 47 - RNA, 30 nt, Artificial Sequence; identical to SEQ ID NO: 12
  unmodified oligo 12

SEQ ID NO: 48 - RNA, 29 nt, Artificial Sequence
  unmodified oligo 13
uagacuuccacagaacucuuggaagucua

SEQ ID NO: 49 - RNA, 33 nt, Artificial Sequence; identical to SEQ ID NO: 9
  unmodified oligo 14

SEQ ID NO: 50 - RNA, 32 nt, Artificial Sequence; identical to SEQ ID NO: 10
  unmodified oligo 15

SEQ ID NO: 51 - RNA, 31 nt, Artificial Sequence; identical to SEQ ID NO: 11
  unmodified oligo 16

SEQ ID NO: 52 - RNA, 30 nt, Artificial Sequence; identical to SEQ ID NO: 12
  unmodified oligo 17

SEQ ID NO: 53 - RNA, 29 nt, Artificial Sequence
  unmodified oligo 18
uagacuuccacagaacucuuggaagucua

SEQ ID NO: 54 - RNA, 25 nt, Artificial Sequence
  unmodified oligo 19
cuuugaagaguucuguggaagucua

SEQ ID NO: 55 - RNA, 25 nt, Artificial Sequence
  unmodified oligo 20
cuuugaagaguucuguggaagucua

SEQ ID NO: 56 - RNA, 19 nt, Artificial Sequence
  unmodified oligo 21
agaguucuguggaagucua

SEQ ID NO: 57 - RNA, 19 nt, Artificial Sequence
  unmodified oligo 22
agaguucacaccaagucua

SEQ ID NO: 58 - RNA, 33 nt, Artificial Sequence; identical to SEQ ID NO: 9
  unmodified oligo 23

SEQ ID NO: 59 - RNA, 32 nt, Artificial Sequence; identical to SEQ ID NO: 10
  unmodified oligo 24

SEQ ID NO: 60 - RNA, 31 nt, Artificial Sequence; identical to SEQ ID NO: 11
  unmodified oligo 25

SEQ ID NO: 61 - RNA, 30 nt, Artificial Sequence; identical to SEQ ID NO: 12
  unmodified oligo 26

SEQ ID NO: 62 - RNA, 29 nt, Artificial Sequence
  unmodified oligo 27
uagacuuccacagaacucuuggaagucua

SEQ ID NO: 63 - RNA, 33 nt, Artificial Sequence; identical to SEQ ID NO: 9
  unmodified oligo 28

SEQ ID NO: 64 - RNA, 32 nt, Artificial Sequence; identical to SEQ ID NO: 10
  unmodified oligo 29

SEQ ID NO: 65 - RNA, 31 nt, Artificial Sequence; identical to SEQ ID NO: 11
  unmodified oligo 30

SEQ ID NO: 66 - RNA, 30 nt, Artificial Sequence; identical to SEQ ID NO: 12
  unmodified oligo 31

SEQ ID NO: 67 - RNA, 29 nt, Artificial Sequence
  unmodified oligo 32
uagacuuccacagaacucuuggaagucua

SEQ ID NO: 68 - RNA, 23 nt, Artificial Sequence
  unmodified oligo 33
uuauagagcaagaacacuguuuu

SEQ ID NO: 69 - RNA, 21 nt, Artificial Sequence
  unmodified oligo 34
aacaguguucuugcucuauaa

SEQ ID NO: 70 - RNA, 19 nt, Artificial Sequence
  unmodified oligo 35
aaccagaagaagcagguga

SEQ ID NO: 71 - RNA, 19 nt, Artificial Sequence
  unmodified oligo 36
ucaccugcuucuucugguu

(19) United States
(12) Patent Application Publication
(10) Pub. No.: US 2021/0332358 A1
(43) Pub. Date: Oct. 28, 2021

(54) MINIATURIZED HAIRPIN RNAi TRIGGERS (mxRNA) AND METHODS OF USES THEREOF

(71) Applicant: Sirnaomics, Inc., Gaithersburg, MD (US)

(72) Inventor: Dmitry SAMARSKY, Westborough, MA (US)

(21) Appl. No.: 17/187,309

(22) Filed: Feb. 26, 2021

Related U.S. Application Data
(63) Continuation of application No. PCT/IB2019/057110, filed on Aug. 23, 2019.
(60) Provisional application No. 62/765,454, filed on Aug. 27, 2018.
(60) Provisional application No. 62/726,619, filed on Sep. 4, 2018.

(51) Int. Cl. C12N 15/113; A61K 9/00

(52) U.S. Cl. CPC C12N 15/113; A61K 9/0019; C12N 2310/14; C12N 2310/531; C12N 2310/122; C12N 2310/343; C12N 2310/313; C12N 2310/321; C12N 2310/3533; C12N 2310/346; C12N 2310/351

(57) ABSTRACT

The present invention relates to novel RNAi triggers that can be chemically synthesized and used to modulate gene expression inside animal cells to study various genes function in laboratories or as an active ingredient for agricultural, veterinary, cosmetic and/or therapeutic applications

The present invention relates to novel RNAi triggers that can be chemically synthesized and used to modulate gene expression inside animal cells to study various genes function in laboratories or as an active ingredient for agricultural, veterinary, cosmetic and/or therapeutic applications

### BACKGROUND

RNA interference or RNAi is a biologic phenomenon characterized by ability of double-stranded RNA molecules to specifically down-regulate individual genes in animals, it was discovered in 1998 by Craig Mello and Andrew Fire, and received a 2006 Nobel Prize for Physiology or Medicine due to its promise to offer novel type of therapeutics. Since 2001, when chemically synthesized RNAi triggers (short interfering RNAs or siRNAs) were shown to work in mammalian cell culture, siRNAs have been extensively used to study various genes functions in research labs around the world. The first RNAi drug Onpattro™ was approved by FDA in August 2018 to help patients with hereditary ATTR amyloidosis. Numerous other RNAi drugs are been currently developed and tested in pre-clinical and clinical studies.

While RNAi promises to become one of the major new drug modality, there are certain challenges associated with it. One of those is the high cost of production of the active ingredient. The conventional RNAi trigger the short interfering RNA (or siRNA) is composed of two 19-25 nt long oligonucleotides (totaling about 40-50 nucleotides) annealed to each other. Production of such molecules requires sophisticated multi-step synthesis, followed, in some cases, by extensive purification procedures, resulting in relatively high production costs. The first RNAi drug Onpattro™ will go for $450,000 per treatment annually, and one of the contributing factors for such a high price is likely to be the drug's cost of production.

### SUMMARY OF THE DISCLOSURE

The terminology used herein is for the purpose of describing particular embodiments only and is not intended to be limiting of the invention. As used herein, the term “and/or” includes any and all combinations of one or more of the associated items. As used herein, the singular forms “a”, “an” and “the” are intended to include the plural forms as well as the singular forms, unless the context clearly indicates otherwise. It will be further understood that the terms “comprises” and/or “comprising” when used in this specification, specify the presence of stated features, steps, operations, elements and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art, to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of features, steps, operations, elements and/or components are disclosed. Each of these has individual benefit and each can also be used in conjunction with one or more, or in some cases all, of the other disclosed features, steps, operations, elements and/or components. Accordingly, for the sake of clarity, this description will refrain from repeating every possible combination of the individual features, steps, operations, elements and/or components in an unnecessary fashion. Nevertheless, the specifications should be read with the understanding that such combinations are entirely within the scope of the invention.

The new miniaturized hairpin RNAi triggers (mxRNA) and methods of uses thereof are discussed herein. In the following description, for the purposes of explanation, numerous specific details are set forth in order to provide a thorough understanding of the present invention. It will be evident, however, to one skilled in the art that the present invention may be practiced without theses specific details.

The present disclosure is to be considered as an exemplification of the invention, and is not intended to limit the invention to the specific embodiments illustrated by the figures or described below.

The current invention will now be described by referencing the appended figures representing certain embodiments. It introduces miniaturized hairpin RNAi trigger molecules (mxRNA) comprising the following components:
1) a 5′ segment of the hairpin stem (segment A);
2) a hairpin loop (segment B);
3) segment of the hairpin stem (segment C);
   where:
1) the total length of (A)+(B)+(C) is 17 to 40 nucleotides;
2) (B) is 0 to 10 nucleotides;
3) (A) is 0-4 nucleotides longer or 0-4 nucleotides shorter than (B)
4) 17 or more nucleotides from the 5′-end of the molecule are complementary to the targeted RNA, e.g. mRNA, IncRNA, and/or other RNA molecules;
   also (optionally) where:
the internal nucleotides in either the single-stranded region or the double-stranded region, or both are chemically modified in sugar and/or base and/or phosphodiester portions of the molecule, e.g. with 2′OMe, 2′F, LNA, PMO, phosphorothioate (PS, PS2), or other chemical modifications, to improve the desired property of the molecule (e.g. to increase stability against the intra- and/or extra-cellular nucleases);
the ends of the molecule are capped or chemically modified, e.g. with vynilphosphonate, inverted nucleotides, or other modifications, to improve the desired property of the molecule (e.g. to increase stability against the intra- and/or extra-cellular nucleases);
mxRNA molecule is conjugated to various delivery moieties, e.g. cholesterol, carbohydrate (GaINAc, other), aptamer, peptide, small molecule, and/or other, to direct and facilitate the extra- and intra-cellular delivery of the molecules.

According to the present invention, there is also provided a conjugate for modulating, preferably inhibiting, expression of a target gene in a cell, said conjugate comprising a nucleic acid attached to one or more ligands, wherein said nucleic acid is preferably not a substrate for dicer, and comprises:

first, second and third nucleic acid portions;

wherein said first portion (i) is at least partially complementary to at least a portion of RNA transcribed from said target gene, and (ii) has a 5′ to 3′ directionality thereby defining 5′ and 3′ regions of said first portion;

wherein said second portion (i) is at least partially complementary to said first portion, and (ii) has a 5′ to 3′ directionality thereby defining 5′ and 3′ regions of said second portion;

wherein said first and second portions dimerise to form an at least partially complementary duplex;

wherein the third nucleic acid portion links the 3′ region of said first portion to the 5′ region of said second portion.

A conjugate according to the present invention comprises a third nucleic acid portion that is at least partially complementary to at least a portion of RNA transcribed from said target gene. Still further, a conjugate according to the present invention can comprise a second nucleic acid portion that is at least partially complementary to at least a portion of RNA transcribed from said target gene.

Preferably, a conjugate according to the present invention comprises one or more ligands that are conjugated to the second nucleic acid portion. Suitably, the one or more ligands are conjugated at the 3 ‘ region of the second nucleic acid portion. Alternatively, the one or more ligands are conjugated at the 3’ region of the first nucleic acid portion and/or at the 5′ region of the second nucleic acid portion. A still further alternative is where the one or more ligands are conjugated at one or more regions intermediate of the 5′ and 3′ regions of the first nucleic acid portion, and/or are conjugated at one or more regions intermediate of the 5′ and 3′ regions of the second nucleic acid portion. As a still further alternative, the one or more ligands are conjugated at one or more regions of the third nucleic acid portion.

Typically, the one or more ligands are any cell directing moiety, such as lipids, carbohydrates, aptamers, vitamins and/or peptides that bind cellular membrane or a specific target on cellular surface. In a preferred embodiment, the one or more ligands comprise one or more carbohydrates, such as a monosaccharide, disaccharide, trisaccharide, tetrasaccharide, oligosaccharide or polysaccharide. Even more preferably, the one or more carbohydrates comprise one or more galactose moieties, one or more lactose moieties, one or more N-Acetyl-Galactosamine moieties, and/or one or more mannose moieties, such as one or more N-Acetyl-Galactosamine moieties, preferably two or three N-Acetyl-Galactosamine moieties.

The one or more ligands can be attached to the nucleic acid in a linear configuration, or in a branched configuration, such that for example the one or more ligands are attached to the nucleic acid as a biantennary or triantennary configuration, or as a configuration based on single ligands at different positions.

A conjugate according to the present invention comprises a nucleic acid that is a single strand that dimerises whereby the first and second portions form an at least partially complementary duplex. Typically, the nucleic acid is 17 to 40 nucleotides in length, preferably at least 20 nucleotides in length, or more preferably is at least 25 nucleotides in length.

In a conjugate according to the present invention, the first nucleic acid portion is 7 to 20 nucleotides in length, preferably 10 to 18 nucleotides in length, more preferably less than 18 nucleotides in length. Similarly, in a conjugate according to the present invention the second nucleic acid portion is 7 to 20 nucleotides in length, preferably 10 to 18 nucleotides in length, more preferably less than 18 nucleotides in length. Still further, it is preferred that in a conjugate according to the present invention the third nucleic acid portion is 1 to 10 nucleotides in length, such as 4 to 9 nucleotides in length, such as 4, 5, 7 or 9 nucleotides in length.

A conjugate according to the present invention further comprises one or more phosphorothioate or phosphorodithioate internucleotide linkages, such as 1 to 15 phosphorothioate or phosphorodithioate internucleotide linkages. Typically, the one or more phosphorothioate or phosphorodithioate internucleotide linkages at one or more of the 5′ and/or 3′ regions of the first and/or second nucleic acid portions. In a preferred embodiment, a conjugate according to the present invention comprises phosphorothioate or phosphorodithioate internucleotide linkages between at least two, preferably at least three, preferably at least four, preferably at least five, preferably at least six, preferably at least seven, preferably at least eight, preferably at least nine, preferably ten, adjacent nucleotides of the third nucleic acid portion, dependent on the number of nucleotides present in the third nucleic acid portion. Still further, a conjugate according to the present invention can comprise a phosphorothioate or phosphorodithioate internucleotide linkage between each adjacent nucleotide that is present in the third nucleic acid portion. Furthermore, a conjugate according to the present invention can comprise a phosphorothioate or phosphorodithioate internucleotide linkage linking the first nucleic acid portion to the third nucleic acid portion and/or the second nucleic acid portion to the third nucleic acid portion.

A conjugate according to the present invention according to the present invention further comprises at least one nucleotide of the first and/or second and/or third nucleic acid portion that is modified. For example, in a conjugate according to the present invention the one or more of the odd numbered nucleotides starting from the 5′ region of the first nucleic acid portion are modified, and/or wherein one or more of the even numbered nucleotides starting from the 5′ region of the first nucleic acid portion are modified, wherein typically the modification of the even numbered nucleotides is a second modification that is different from the modification of odd numbered nucleotides. Typically, the one or more of the odd numbered nucleotides starting from the 3′ region of the second nucleic acid portion are modified by a modification that is different from the modification of odd numbered nucleotides of the first nucleic acid portion.

Further characteristics of the modification pattern can be as follows, but for the avoidance of doubt the following statements are not limiting on the scope of the invention as described herein:
one or more of the even numbered nucleotides starting from the 3′ region of the second nucleic acid portion are modified by a modification that is different from the modification of odd numbered nucleotides of the second nucleic acid portion; and/or
at least one or more of the modified even numbered nucleotides of the first nucleic acid portion is adjacent to at least one or more of the differently modified odd numbered nucleotides of the first nucleic acid portion; and or
at least one or more of the modified even numbered nucleotides of the second nucleic acid portion is adjacent to at least one or more of the differently modified odd numbered nucleotides of the second nucleic acid portion; and/ar
a plurality of adjacent nucleotides of the first nucleic acid portion are modified by a common modification; and or
a plurality of adjacent nucleotides of the second nucleic acid portion are modified by a common modification; and/or
the plurality of adjacent commonly modified nucleotides are 2 to 4 adjacent nucleotides, preferably 3 or 4 adjacent nucleotides, so that typically the plurality of adjacent commonly modified nucleotides are located in the 5′ region of the second nucleic acid portion, when the third nucleic acid portion links the 3′ region of said first portion to the 5′ region of said second portion; and/or
the plurality of adjacent commonly modified nucleotides is located in third nucleic acid region; and or
a plurality of odd numbered nucleotides of the first and/or second nucleic acid portions are modified, such that typically the plurality of odd numbered nucleotides are modified by a common modification; and/or
a plurality of even numbered nucleotides of the first and/or second nucleic acid portions are modified by a second modification, such that typically the plurality of even numbered nucleotides are modified by a common second modification; and/or
the one or more of the modified nucleotides of the first nucleic acid portion do not have a common modification present in the corresponding nucleotide of the second nucleic acid portion of the duplex; and/or
the one or more of the modified nucleotides of the first nucleic acid portion are shifted by at least one nucleotide relative to a commonly modified nucleotide of the second nucleic acid portion.

Typically, in a conjugate according to the present invention the modification and/or modifications are each and individually sugar, backbone or base modifications, and are suitably selected from the group consisting of 3′-terminal deoxy-thymine, 2′-O-methyl, a 2′-deoxy-modification, a 2′-amino-modification, a 2′-alkyl-modification, a morpholino modification, a phosphoramidate modification, phosphorothioate or phosphorodithioate group modification, a 5′ phosphate or 5′ phosphate mimic modification and a cholesteryl derivative or a dodecanoic acid bisdecylamide group modification.

The modification can be any one of a locked nucleotide; an abasic nucleotide or a non-natural base comprising nucleotide.

In a preferred embodiment, at least one modification is 7-O-methyl. In a further preferred embodiment, at least one modification is 2′-F.

In a further preferred embodiment, the nucleotides at any of positions 2 and 14 downstream from the first nucleotide of the 5′ region of the first nucleic acid portion do not contain 2′-O-methyl modifications in ribose moieties, and/or the nucleotides of the second nucleic acid portion, that correspond in position to any of the nucleotides of the first nucleic acid portion at any of positions 9 to 11 downstream from the first nucleotide of the 5′ region of the first nucleic acid portion, do not contain 2′-O-methyl modifications in ribose moieties.

A conjugate according to the present invention preferably further comprises one or more unmodified nucleotides, which can typically replace any modified nucleotide as hereinbefore described. Such one or more unmodified nucleotides can be positioned in the 5′ region of the second nucleic acid portion and/or can be positioned in the third nucleic acid portion at positions proximal to the second nucleic acid portion.

Preferably, the one or more, preferably one, unmodified nucleotide represent the nucleotide or nucleotides of the 5′ region of the second nucleic acid portion, typically the nucleotide of the second nucleic acid portion that is directly linked to the third nucleic acid portion, and; or the nucleotide or nucleotides of the third nucleic acid portion proximal the 5′ region of the second nucleic acid portion, typically the nucleotide of the third nucleic acid portion that is directly linked to the second nucleic acid portion, and preferably represent any of the nucleotides at any of positions 17, 18, 19, 20, 21, 22, 23, 24 and/or 25 downstream from the first nucleotide of the 5′ region of the first nucleic acid portion, preferably positions 18, 19, 20 and/or 21.

In a conjugate according to the present invention, typically all nucleotides other than the unmodified nucleotides, and f or the nucleotides at any of positions 2 and 14 downstream from the first nucleotide of the 5′ region of the first nucleic acid portion, and/or the nucleotides of the second nucleic acid portion, that correspond in position to any of the nucleotides of the first nucleic acid portion at any of positions 9 to 11 downstream from the first nucleotide of the 5′ region of the first nucleic acid portion, contain 2′-O-methyl modifications in ribose moieties

In a preferred embodiment, all odd numbered nucleotides of the first nucleic acid region, starting from the 5′ region of the first nucleic acid portion, are 2′-0-methyl modified, and all even numbered nucleotides of the first nucleic acid region, starting from the 5′ region of the first nucleic acid portion, are 2′-F modified.

In a particular embodiment, other than the unmodified nucleotide or nucleotides of the second nucleic acid portion, all odd numbered nucleotides of the second nucleic acid region, starting from the 3′ region of the second nucleic acid portion, are 2′-F modified, and all even numbered nucleotides of the second nucleic acid region, starting from the 3′ region of the second nucleic acid portion, are 2′-0-methyl modified. For example, a plurality of adjacent commonly modified nucleotides of 2 to 4 adjacent nucleotides; preferably 3 or 4 adjacent nucleotides, are located downstream of the unmodified nucleotide or nucleotides of the second nucleic acid portion, and for the remaining nucleotides of the second nucleic acid portion all odd numbered nucleotides of the second nucleic acid region, starting from the 3′ region of the second nucleic acid portion, are 2′-F modified, and all even numbered nucleotides of the second nucleic acid region, starting from the 3′ region of the second nucleic acid portion, are 2′-0-methyl modified.

In a further embodiment of the present invention, in a conjugate as described herein the nucleotides in the third nucleic acid portion are modified in an alternating 2′-0-methyl, 2′-F, pattern, starting with a 2′-0-methyl modification adjacent to the 3′ region of the first nucleic acid portion.

A conjugate according to the present invention can further comprise at least one vinylphosphonate modification, such as at least one vinylphosphonate modification in the 5′ region of the first nucleic acid portion.

In a conjugate according to the present invention, one or more nucleotides of at least one of the first nucleic acid portion and the second nucleic acid portion is an inverted nucleotide and is attached to the adjacent nucleotide via the 3′ carbon of the nucleotide and the 3′ carbon of the adjacent nucleotide, and or one or more nucleotides of at least one of the first nucleic acid portion and the second nucleic acid portion is an inverted nucleotide and is attached to the adjacent nucleotide via the 5′ carbon of the nucleotide and the 5′ carbon of the adjacent nucleotide

A conjugate according to the present invention can further comprise one or more nucleotides at the 3′ region of at least one of the first nucleic acid portion and the second nucleic acid portion is an inverted nucleotide and is attached to the adjacent nucleotide via the 3′ carbon of the terminal nucleotide and the 3′ carbon of the adjacent nucleotide, and/or one or more nucleotides at the 5′ region of at least one of the first nucleic acid portion and the second nucleic acid portion is an inverted nucleotide and is attached to the adjacent nucleotide via the 5′ carbon of the terminal nucleotide and the 5′ carbon of the adjacent nucleotide, and or one or more nucleotides intermediate the 3′ and 5′ regions of at least one of the first nucleic acid portion and the second nucleic acid portion is an inverted nucleotide and is attached to the adjacent nucleotide via the 3′ carbon of the terminal nucleotide and the 3′ carbon of the adjacent nucleotide and/or one or more nucleotides intermediate the 3′ and 5′ regions of at least one of the first nucleic acid portion and the second nucleic acid portion is an inverted nucleotide and is attached to the adjacent nucleotide via the 5′ carbon of the terminal nucleotide and the 5′ carbon of the adjacent nucleotide, and/or one or more nucleotides of at least one of the third nucleic acid portion is an inverted nucleotide and is attached to the adjacent nucleotide via the 3′ carbon of the terminal nucleotide and the 3′ carbon of the adjacent nucleotide and/or one or more nucleotides of at least one of the third nucleic acid portion is an inverted nucleotide and is attached to the adjacent nucleotide via the 5′ carbon of the terminal nucleotide and the 5′ carbon of the adjacent nucleotide. Typically, the 3′ and/or 5′ inverted nucleotide of the first and/or second strand is attached to the adjacent nucleotide via a phosphate group by way of a phosphodiester linkage; or the 3′ and/or 5′ inverted nucleotide of the first and/or second strand is attached to the adjacent nucleotide via a phosphorothioate group; or the 3′ and/or 5′ inverted nucleotide of the first and/or second strand is attached to the adjacent nucleotide via a phosphorodithioate group.

A conjugate according to the present invention can be blunt ended at one end. Alternatively, a conjugate according to the present invention can comprise a first or second nucleic acid portion that has an overhang.

According to the present invention, there is further provided a homo-dimer RNA molecule comprising two nucleic acid molecules as hereinbefore described, wherein the nucleic acid molecules are bound together through complementary interactions, where the first portion of the first molecule interacts with the second portion of the second molecule and there is a third portion in each molecule that generates a bulge structure intermediate of the first and second portions of the respective nucleic acid molecules.

A conjugate or homo-dimer RNA molecule and/or conjugate as described herein is directed at a target RNA that is selected from at least one of: mRNA, IncRNA, and/or other RNA molecules.

The present invention further comprises:
A composition comprising a conjugate or molecule as described herein, and a physiologically acceptable excipient;
A conjugate or molecule as described herein, for use in the treatment of a disease or disorder;
Use of a conjugate or molecule as described herein, in the manufacture of a medicament for treating a disease or disorder;
A method of treating a disease or disorder comprising administration of a conjugate or molecule as described herein, to an individual in need of treatment, for example by administration subcutaneously or intravenously to the individual;
Use of a conjugate or molecule as described herein, for use as a cosmetic;
Use of a conjugate or molecule as described herein, for use in research as gene function analysis tool;
A process of making a conjugate as described herein.

There is also provided by the present invention a conjugate as hereinbefore described wherein the conjugate comprises a sequence selected from the group consisting of SEQ ID NOs: 14, 15, 16, 17 and 18, the linker and the tri-valent GaINAc moiety being at the 3′-end of the nucleic acid moiety. For each of sequences of SEQ ID NOs: 14, 15, 16, 17 and 18, these comprise first, second and third nucleic acid portions as follows:
wherein said first portion starts at the beginning of each SEQ ID NO and (i) is at least partially complementary to at least a portion of RNA transcribed from said target gene (MAP4K4 in this instance), and (ii) has a 5′ to 3′ directionality thereby defining 5′ and 3′ regions of said first portion;
wherein said second portion (i) is at least partially complementary to said first portion, and (ii) has a 5′ to 3″ directionality thereby defining 5′ and 3′ regions of said second portion;
wherein the third nucleic acid portion links the 3′ region of said first portion to the 5′ region of said second portion,
   and wherein:
For SEQ ID NO 14: The first portion comprises 14 nucleotides, the second portion comprises 14 nucleotides and the third portion comprises 5 nucleotides;
For SEQ ID NO 15: The first portion comprises 14 nucleotides, the second portion comprises 14 nucleotides and the third portion comprises 4 nucleotides;
For SEQ ID NO 16: The first portion comprises 12 nucleotides, the second portion comprises 12 nucleotides and the third portion comprises 7 nucleotides;
For SEQ ID NO 17: The first portion comprises 13 nucleotides, the second portion comprises 13 nucleotides and the third portion comprises 4 nucleotides;
For SEQ ID NO 18: The first portion comprises 10 nucleotides, the second portion comprises 10 nucleotides and the third portion comprises 9 nucleotides.

For the sequences as set out above, the first and second portions dimerise to form an at least partially complementary duplex as hereinbefore described.

Typically such sequences comprise phosphorothioate or phosphorodithioate internucleotide linkages between each of the nucleotides of the third nucleic acid portion thereof and or unmodified nucleotides in positions 17, 18, 19, 20, 21, 22, 23, 24 and/or 25 from the 5′ region of the first portion.

### BRIEF DESCRIPTION OF THE DRAWINGS

FIG. 1 shows schematic examples of the miniaturized hairpin RNAi triggers (mxRNA™). In each example, the segment A represent the 5′ portion of the hairpin's duplex, the segment B represents the single-stranded loop for the hairpin, and the segment C represents the 3′ portion of the hairpin's duplex. Thick lines represent sequence complementary to a corresponding sequence of the targeted RNA transcript.

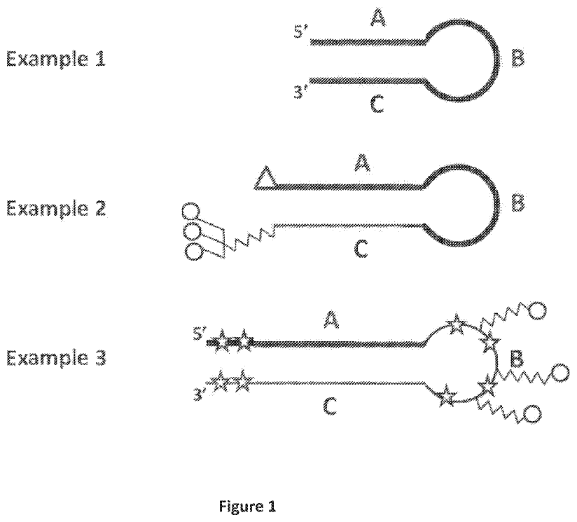

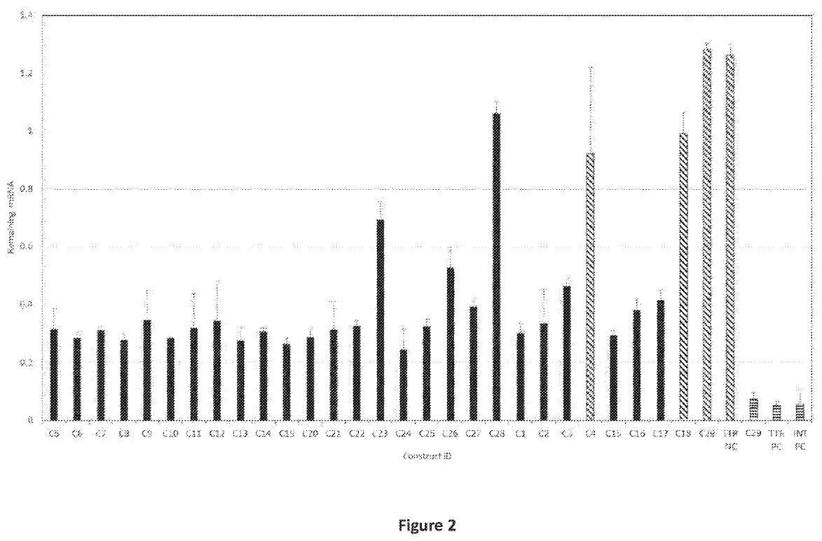

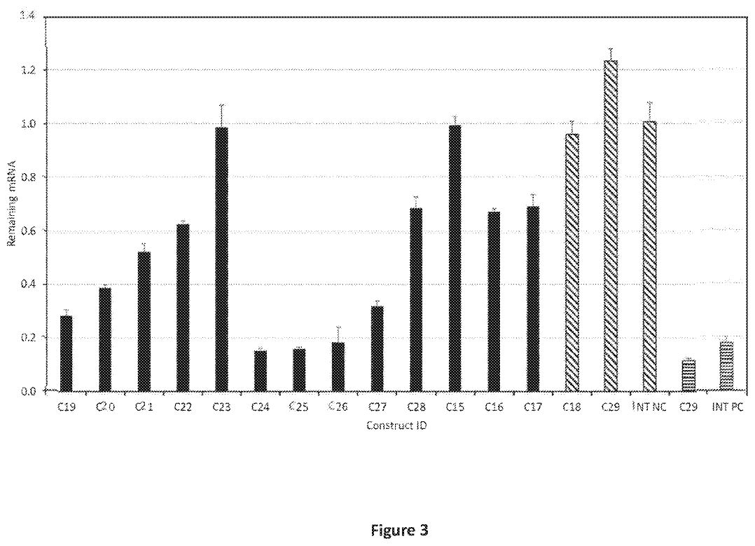

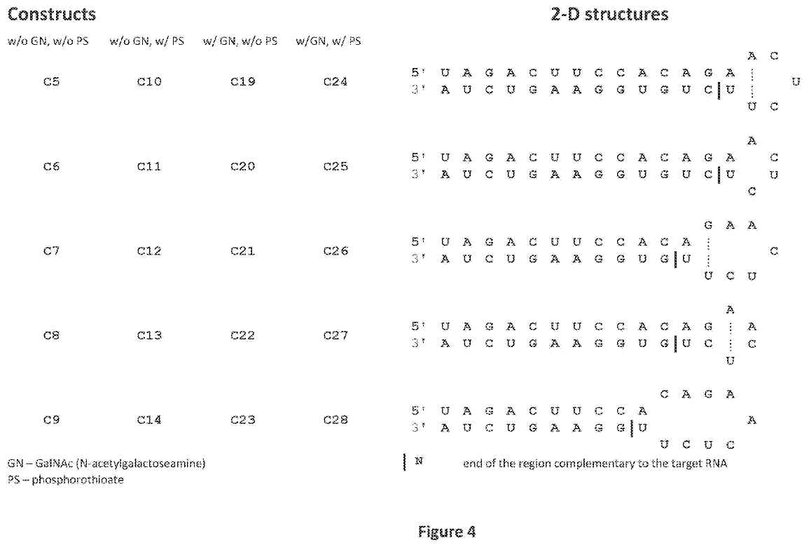

Example 1 of FIG. 1 shows schematics for one of the smallest possible mxRNA, in which segment A is 7 nucleotides, segment B is 4 nucleotides, segment C is 7 nucleotides and all 18 nucleotides are complementary to the targeted RNA.

Example 2 of FIG. 1 shows schematics for an mxRNA, in which segment A is 14 nucleotides, segment B is 4 nucleotides, segment C is 14 nucleotides (32 nucleotides in total) and 18 nt from the 5′-end of the molecule are complementary to the targeted RNA (thick line) and the triangle at the 5′ end of the molecule represents a chemical moiety, such as a cap, for example vinylphosphonate to increase resistance against nucleases and a trivalent chemical moiety (lines and circles), for example GaINAc, is conjugated via linker (wiggled line) to the 3′-end of the molecule to facilitate delivery of the molecule to the cells, for example hepatocytes in vitro and/or in vivo.

Example 3 of FIG. 1 shows schematics for an mxRNA, in which segment A is 18 nucleotides, segment B is 4 nucleotides, segment C is 18 nucleotides (40 nucleotides in total) and first 18% nucleotides from the 5′-end of the molecule are complementary to the targeted RNA (thick line) and entire molecule is chemically modified with sugar modifications, for example 2′OMe and/or 2′F (not shown) to increase nuclease stability and 2 nucleotides on each end of the molecule and nucleotides in the loop are modified in the phosphodiester positions, for example phosphorothioates (stars), to increase nuclease stability, and delivery conjugate moieties (e.g. GaINAc, cholesterol, other) are attached to the single-strand loop region of the molecule.

FIG. 1 depicts three examples of the mxRNA molecules as described above. The stem-and-loop configuration in Example 1 exemplifies one of the smallest versions of the mxRNA 18 nucleotides long. Essentially the entire sequence of the molecule is complementary to the targeted RNA. It is understood that in such particular nearly extreme case, the targeted sequence would have to possess relatively long (7 nucleotides) palindromic sequences separated by 4 nucleotides. Example 3, in contrast depicts molecule with one of the largest (40 nucleotides in total) mxRNA stem-and-loop configurations. Such configuration is akin to the structure of the conventional shRNAs, with the important difference that it most likely would not be processed inside the cells by the Dicer enzyme (due to single-stranded region in the place where the Dicer would be expected to cleave and/or due to the chemical modifications that would likely be used to stabilise the molecule against endonucleases) to produce conventional siRNA molecules. Example 2 depicts an intermediate (between configuration depicted in Example 1 and Example 3) version of the mxRNA stem-and-loop configuration. Such configurations pose no constrain on the targeting sequence, yet is much more compact than conventional siRNAs and shRNAs. It is understood that numerous other permutations in the stem-and-loop configuration design are possible.

FIG. 1, in particular the Examples 2 and 3, also depicts where certain chemical modifications and conjugations can be applied. In particular, any nucleotide (in sugar, base and/or phosphodiester linkage) of the internal backbone of the molecule can be modified with various chemical modification to improve the properties of the molecule (e.g. to increase stability against the intra- and extra-cellular nucleases). In addition, the ends of the molecules can be further enhanced by the cap structures and chemical modifications. Various nucleic acid and non-nucleic acid moieties can be also conjugated to the various parts of the mxRNA to add additional properties (e.g. enhanced and/or targeted delivery capabilities).

FIGS. 2 and 3 present the graphs for the results of the experiments described in the Examples section of the application (below).

FIG. 4 depicts the secondary (2D) structures of the mxRNA molecules used in experiments described in the Examples section of the application (below).

mxRNA molecules can be chemically synthesized using conventional and/or advanced approaches, and be used as research tools to study various genes functions in the labs, and/or as active ingredients for agricultural, veterinary, cosmetic and/or therapeutic applications.

Aspects of the invention are demonstrated by the following non-limiting examples.

### EXAMPLES

### Example 1: Single Dose Transfection in AML-12 Cells

Activity tests for mxRNAs versus conventional double stranded siRNA constructs that were directed against MAP4K4 were conducted. Hep3B cells were incubated in 96-well plates at a density of 15,000 cells per each well. The compounds tested with this study were at a final concentration of 50 nM. Reverse transfection was carried out using RNAiMax at 0.3 μL per well. In addition to the test compounds two controls ((TTR PC) TTR-directed siRNA and (INT PC) aha-1 directed siRNA) were also used (Tables 3 & 4). The duration of incubation was 24 hours. Subsequently mRNA was isolated and quantified using a bDNA assay (Quantigene 1.0/2.0). The readouts were normalised to GAPDH transcript, and the mean of quadruplicates was determined. The values from mock treated cells was set at 1.

A summary of the results obtained from this experiment are presented in Table 1 and FIG. 2.

TABLE 1
Summary of results for Example 1

| | | |
|---|---|---|
| SEQ ID | construct | remaining mRNA |

| NO(s). | ID | mean | SD |
|---|---|---|---|
| 9 | C5 | 0.31 | 0.07 |
| 10 | C6 | 0.28 | 0.01 |
| 11 | C7 | 0.31 | 0.01 |
| 12 | C8 | 0.28 | 0.02 |
| 13 | C9 | 0.35 | 0.10 |
| 14 | C10 | 0.28 | 0.00 |
| 15 | C11 | 0.32 | 0.12 |
| 16 | C12 | 0.34 | 0.14 |
| 17 | C13 | 0.28 | 0.04 |
| 18 | C14 | 0.31 | 0.01 |
| 23 | C19 | 0.26 | 0.02 |
| 24 | C20 | 0.29 | 0.03 |
| 25 | C21 | 0.31 | 0.10 |
| 26 | C22 | 0.33 | 0.02 |
| 27 | C23 | 0.70 | 0.06 |
| 28 | C24 | 0.24 | 0.07 |
| 29 | C25 | 0.33 | 0.02 |
| 30 | C26 | 0.53 | 0.07 |
| 31 | C27 | 0.39 | 0.02 |
| 32 | C28 | 1.06 | 0.04 |
| 1, 2 | C1 | 0.30 | 0.03 |
| 3, 4 | C2 | 0.33 | 0.12 |
| 5, 6 | C3 | 0.46 | 0.02 |
| 7, 8 | C4− | 0.92 | 0.30 |
| 1, 19 | C15 | 0.29 | 0.02 |
| 3, 20 | C16 | 0.38 | 0.03 |
| 5, 21 | C17 | 0.41 | 0.04 |
| 7, 22 | C18− | 0.99 | 0.07 |
| 33, 34 | C29− | 1.28 | 0.02 |
| XD-12171− | TTR NC− | 1.26 | 0.04 |
| 33, 34 | C29+ | 0.07 | 0.02 |
| XD-12171+ | TTR PC+ | 0.05 | 0.01 |
| XD-00033+ | INT PC+ | 0.06 | 0.05 |

“−” Denotes a negative control and “+” denotes a positive control

### Example 2: Single Dose Direct Incubation of GaINAc-Conjugated Compounds in Primary Hepatocytes

Primary mouse hepatocytes (Lot#MC830; ThermoFisher Scientific) were incubated in a 96-well plate at a density of 60,000 cells per well. The compounds tested with this study were added at a final concentration of 500 nM. In addition to the test compounds two controls ((XD-12171) TTR-directed siRNA and (XD-00033) aha-1 directed siRNA as a negative control) were also used (Tables 3 & 4). A direct incubation transfection (without transfection lipid) method was used. The duration of incubation was 72 hours. Subsequently mRNA was isolated and quantified using a bDNA assay (Quantigene 1.0/2.0). The readouts were normalised to GAPDH, and the mean of quadruplicates was determined. The values from mock treated cells was set at 1.

A summary of the results obtained from this experiment are presented in Table 2 and FIG. 3.

TABLE 2
Summary of results for Example 2

| Description | construct | SEQ ID | remaining mRNA | |
|---|---|---|---|---|
| of construct | ID | NO(s). | mean | SD |
| w/o phosphorothioate | C19 | 23 | 0.28 | 0.02 |
| | C20 | 24 | 0.39 | 0.01 |
| | C21 | 25 | 0.52 | 0.03 |
| | C22 | 26 | 0.63 | 0.01 |
| | C23 | 27 | 0.99 | 0.08 |
| with phosphorothioate | C24 | 28 | 0.15 | 0.01 |
| | C25 | 29 | 0.16 | 0.01 |
| | C26 | 30 | 0.18 | 0.06 |
| | C27 | 31 | 0.32 | 0.02 |
| | C28 | 32 | 0.69 | 0.04 |
| duplex non-stabilized | C15 | 1, 19 | 0.99 | 0.03 |
| duplex stable V1 (PC) | C16 | 3, 20 | 0.67 | 0.01 |
| duplex stable V2 (PC) | C17 | 5, 21 | 0.69 | 0.04 |
| NC1 (flipped central part) | C18− | 7, 22 | 0.96 | 0.05 |
| NC2 (targeting TTR) | C29− | 33, 34 | 1.23 | 0.05 |
| NC3 (no GalNAc) | INT NC− | XD-00033− | 1.01 | 0.07 |
| PC1 ALNY TTR | C29+ | 33, 34 | 0.12 | 0.01 |
| PC2 AXO TTR | INT PC+ | XD-12171+ | 0.18 | 0.02 |

“−” Denotes a negative control and “+” denotes a positive control

Summary of Results from Examples 1 and 2

The results confirm that the single-oligo miniaturized hairpin structures (mxRNA) can elicit target gene knock-down, if used with transfection reagent and unobstructed with conjugate moieties, as was previously demonstrated in Lapierre et al, 2011.

We demonstrated that mxRNA molecules conjugated with a bulky chemical moiety (GaINAc in this case) can still elicit target gene knock-down, when used with a transfection reagent. This is a new and non-trivial finding since adding a conjugate to the 3′ end of the active strand could have affected the mxRNAs' ability to be recognized by the RNAi machinery, to enter an RNA-induced silencing complex (RISC) and/or to remain active within the RISC.

Next, mxRNA-conjugates (conjugated with GaINAc in this case) were demonstrated to enter cells via receptor-mediated uptake and to yield activities higher than those of conventional siRNA targeting exactly the same portion of mRNA (e.g. mxRNA C24, C25, C26 constructs compared with conventional C16, C17 constructs). This is a new finding and without wishing to be bound to a particular theory, such improvement could be due to the smaller size of the mxRNA-conjugate molecules (approximately 32 nucleotides in total), if compared with conventional siRNAs (approximately 42 nucleotides in total).

Finally the results showed that the use of diverse chemical modification patterns comprising phosphodiester linkage modifications (e.g. phosphorothioate modifications) and/or sugar modifications (e.g. 2′OH positions) can further improved the performance of mxRNAs.

TABLE 3
Single-stranded mxRNA constructs used in this study

| con- | SEQ | | Experiment | |
|---|---|---|---|---|
| struct | ID | | | |
| id | NO.(s) | Sequence | type | Target |
| C5 | 9 | puAfgAfcUfuCfcAfcAfgAfaCfuCfuuCfUfGfuGfgAfaGfuCfuAf | Transfection | mmMAP4K4 |
| C6 | 10 | puAfgAfcUfuCfcAfcAfgAfaCfuCfuCfUfGfuGfgAfaGfuCfuAf | Transfection | mmMAP4K4 |
| C7 | 11 | puAfgAfcUfuCfcAfcAfgAfaCfuCfUfUfGfuGfgAtaGfuCfuAf | Transfection | mmMAP4K4 |
| C8 | 12 | puAfgAtcUfuCfcAfcAfgAfaCfuCfUfGfuGfgAfaGfuCfuAf | Transfection | mmMAP4K4 |
| C9 | 13 | puAfgAfcUfuCfcAfcAfgAfaCfUfCfUfuGfgAfaGfuCfuAf | Transfection | mmMAP4K4 |
| C10 | 14 | puAfgAfcUfuCfcAfcAfgAfsasCfsusCfsusUCfUfGfuGfgAfaGfuCfuAf | Transfection | mmMAP4K4 |
| C11 | 15 | puAfgAfcUfuCfcAfcAfgAfsasCfsusCfsUCfUfGfuGfgAfaGfuCfuAf | Transfection | mmMAP4K4 |
| C12 | 16 | puAfgAfcUfuCfcAfcAfsgsAfsasCfsusCfsUfsUGfuGfgAfaGfuCfuAf | Transfection | mmMAP4K4 |
| C13 | 17 | puAfgAfcUfuCfcAfcAfgsAfsasCfsusCfsUGfuGfgAfaGfuCfuAf | Transfection | mmMAP4K4 |
| C14 | 18 | puAfgAfcUfuCfcAfscsAfsgsAfsasCfsUfsCfsUfsUGfgAfaGfuCfuAf | Transfection | mmMAP4K4 |
| C19 | 23 | puAfgAfcUfuCfcAfcAfgAfaCfuCfuuCfUfGfuGfgAfaGfuCfuAf(NHC6) | Transfection & | mmMAP4K4 |
| | | (GalNAc3) | incubation | |
| C20 | 24 | puAfgAfcUfuCfcAfcAfgAfaCfuCfuCfUfGfuGfgAfaGfuCfuAf(NHC6) | Transfection & | mmMAP4K4 |
| | | (GalNAc3) | incubation | |
| C21 | 25 | puAfgAfcUfuCfcAfcAfgAfaCfuCfUfUfGfuGfgAfaGfuCfuAf(NHC6) | Transfection & | mmMAP4K4 |
| | | (GalNAc3) | incubation | |
| C22 | 26 | puAfgAfcUfuCfcAfcAfgAfaCfuCfUfGfuGfgAfaGfuCfuAf(NHC6) | Transfection & | mmMAP4K4 |
| | | (GalNAc3) | incubation | |
| C23 | 27 | puAfgAfcUfuCfcAfcAfgAfaCfUfCfUfuGfgAfaGfuCfuAf(NHC6)(GalNAc3) | Transfection & | mmMAP4K4 |
| | | | incubation | |
| C24 | 28 | puAfgAtUfuCfcAfcAfgAfsasCfsusCfsusUCfUfGfuGfgAfaGfuCfuAf | Transfection & | mmMAP4K4 |
| | | (NHC6)(GalNAc3) | incubation | |
| C25 | 29 | puAfgAfcUfuCfcAfcAfgAfsasCfsusCfsUCfUfGfuGfgAfaGfuCfuAf(NHC6) | Transfection & | mmMAP4K4 |
| | | (GalNAc3) | incubation | |
| C26 | 30 | puAfgAfcUfuCfcAfcAfsgsAfsasCfsusCfsUfsUGfuGfgAfaGfuCfuAf | Transfection & | mmMAP4K4 |
| | | (NHC6)(GalNAc3) | incubation | |
| C27 | 31 | puAfgAfcUfuCfcAfcAfgsAfsasCfsusCfsUGfuGfgAfaGfuCfuAf(NHC6) | Transfection & | mmMAP4K4 |
| | | (GalNAc3) | incubation | |
| C28 | 32 | puAfgAfcUfuCfcAfscsAfsgsAfsasCfsUfsCfsUfsUGfgAfaGfuCfuAf | Transfection & | mmMAP4K4 |
| | | (NHC6)(GalNAc3) | incubathon | |

TABLE 4
Conventional duplex siRNA constructs used in this study

| Con- | SEQ | | SEQ | | |
|---|---|---|---|---|---|
| struct | ID | | ID | | |
| ID | NO. | antisense sequence | NO. | sense sequence | target |
| 1* | 1 | pUAGACUUCCACAGAACUCUUCAAAG | 2 | cuuugaagaguuCUGuggaagucua | mmMAP4K4 |
| 2* | 3 | pUAGACUUCCACAGAACUCUUCAAAG | 4 | cuuugaagaguuCUGuggaagucua(NHC6)(GalNAc3) | mmMAP4K4 |
| 3* | 5 | puAfgAfcUfuCfcAfcAfgAfaCfuCfu | 6 | CfuUfuGfaAfgAfgUfuCfUfGfuGfgAfaGfuCfuAf | mmMAP4K4 |
| | | UfcAfaAfg | | | |
| 4*- | 7 | puAfgAfcUfuCfcAfcAfgAfaCfuCfu | 8 | CfuUfuGfaAfgAfgUfuCfUfGfuGfgAfaGfuCfuAf | mmMAP4K4 |
| | | UfcAfaAfg | | (NHC6)(GalNAc3) | |
| 15# | 1 | puAfgAfcUfuCfcAfcAfgAfaCfuCfu | 19 | AfgAfgUfuCfUfGfuGfgAfaGfuCfuAf | mmMAP4K4 |
| 16# | 3 | puAfgAfcUfuCfcAfcAfgAfaCfuCfu | 20 | AfgAfgUfuCfUfGfuGfgAfaGfuCfuAf(NHC6)(GalNAc3) | mmMAP4K4 |
| 17# | 5 | puAfgAfcUfuGfgUfgUfgAfaCfuCfu | 21 | AfgAfgUfuCfAfCfaCfcAfaGfuCfuAf | mmMAP4K4 |
| 18#- | 7 | puAfgAfcUfuGfgUfgUfgAfaCfuCfu | 22 | AfgAfgUfuCfAfCfaCfcAfaGfuCfuAf(NHC6)(GalNAc3) | mmMAP4K4 |
| 29#- | 33 | puUfaUfaGfaGfcAfaGfaAfcAfcUfg | 34 | AsaCsaGsuGsuUscUsuGscUscUsaUsaAf(NHC6) | mmTTR |
| | | UfusUfsu | | (GalNAc3) | |
| INT PC- | 35 | usUfsaUfaGfaGfcAfagaAfcAfcUfg | 36 | AfsasCfaGfuGfuUfCfUfuGfcUfcUfaUfaAf(NHC6) | mmTTR |
| | | Ufususu | | (GalNAc3) | |
| INT PC- | | | | | Aha1 |

*denotes that the duplex construct was subjected to transfection only; # denotes that the duplex construct was subjected to transfection and incubation experiments; -denotes that the duplex was used as a control.
Table 3 and 4 keys
p = phosphate
u, a, c, g = 2′-methyl modified
Uf, Af, Cf, Gf = 2′-fluoro modified
U, A, C, G = unmodified
s = phosphorothioate
(NHC6) = linker
(GalNAc3) = trivalent N-acetylgalactosamine

1. A conjugate for modulating, preferably inhibiting, expression of a target gene in a cell, said conjugate comprising a nucleic acid attached to one or more ligands, wherein said nucleic acid is preferably not a substrate for dicer, and comprises:
first, second and third nucleic acid portions;
wherein said first portion (i) is at least partially complementary to at least a portion of RNA transcribed from said target gene, and (ii) has a 5′ to 3′ directionality thereby defining 5′ and 3′ regions of said first portion;
wherein said second portion (i) is at least partially complementary to said first portion, and (ii) has a 5′ to 3′ directionality thereby defining 5′ and 3′ regions of said second portion;
wherein said first and second portions dimerise to form an at least partially complementary duplex;
wherein the third nucleic acid portion links the 3′ region of said first portion to the 5′ region of said second portion.

2. A conjugate according to claim 1, wherein said third nucleic acid portion is at least partially complementary to at least a portion of RNA transcribed from said target gene.

3. A conjugate according to claim 1, wherein said second nucleic acid portion is at least partially complementary to at least a portion of RNA transcribed from said target gene.

4. A conjugate according to claim 1, wherein said one or more ligands are conjugated to said second nucleic acid portion.

5. A conjugate according to claim 4, wherein said one or more ligands are conjugated at the 3 region of the second nucleic acid portion.

6. A conjugate according to claim 1, wherein said one or more ligands are conjugated at one or more regions intermediate of the 5′ and 3′ regions of the first nucleic acid portion.

7. A conjugate according to claim 1, wherein said one or more ligands are conjugated at one or more regions intermediate of the 5′ and 3′ regions of the secondnucleic acid portion.

8. A conjugate according to claim 1, wherein said one or more ligands are conjugated at one or more regions of the third nucleic acid portion.

9. A conjugate according to claim 1, wherein said one or more ligands are any cell directing moiety, such as lipids, carbohydrates, aptamers, vitamins and/or peptides that bind cellular membrane or a specific target on cellular surface.

10. A conjugate according to claim 9, wherein said one or more ligands comprise one or more carbohydrates.

11. A conjugate according to claim 10, wherein said one or more carbohydrates can be a monosaccharide, disaccharide, trisaccharide, tetrasaccharide, oligosaccharide or polysaccharide.

12. A conjugate according to claim 11, wherein said one or more carbohydrates comprise one or more galactose moieties, one or more lactose moieties, one or more N-Acetyl-Galactosamine moieties, and/or one or more mannose moieties.

13. A conjugate according to claim 12, wherein said one or more carbohydrates comprise one or more N-Acetyl-Galactosamine moieties.

14. A conjugate according to claim 13, which comprises two or three N-Acetyl-Galactosamine moieties.

15. A conjugate according to claim 1, wherein said one or more ligands are attached to said nucleic acid in a linear configuration, or in a branched configuration.

16. A conjugate according to claim 15, wherein said one or more ligands are attached to said nucleic acid as a biantennary or triantennary configuration, or as a configuration based on single ligands at different positions.

17. A conjugate according to claim 1, wherein said nucleic acid is a single strand that dimerises whereby said first and second portions form said at least partially complementary duplex.

18. A conjugate according to claim 1, wherein said nucleic acid is 17 to 40 nucleotides in length.

19. A conjugate according to claim 18, wherein said nucleic acid is at least 20 nucleotides in length, or more preferably is at least 25 nucleotides in length.

20. A conjugate according to claim 1, wherein said first nucleic acid portion is 7 to 20 nucleotides in length, preferably 10 to 18 nucleotides in length, more preferably less than 18 nucleotides in length.

21. A conjugate according to claim 1, wherein said second nucleic acid portion is 7 to 20 nucleotides in length, preferably 10 to 18 nucleotides in length, more preferably less than 18 nucleotides in length.

22. A conjugate according to claim 1, wherein said third nucleic acid portion is 1 to 10 nucleotides in length, such as 4 to 9 nucleotides in length, such as 4, 5, 7 or 9 nucleotides in length.

23. A conjugate according to claim 1, which comprises one or more phosphorothioate or phosphorodithioate internucleotide linkages.

24. A conjugate according to claim 23, which comprises 1 to 15 phosphorothioate or phosphorodithioate internucleotide linkages.

25. A conjugate according to claim 23, which comprises one or more phosphorothioate or phosphorodithioate internucleotide linkages at one or more of the 5′ and/or 3′ regions of the first and/or second nucleic acid portions.

26. A conjugate according to claim 23, which comprises phosphorothioate or phosphorodithioate internucleotide linkages between at least two, preferably at least three, preferably at least four, preferably at least five, preferably at least six, preferably at least seven, preferably at least eight, preferably at least nine, preferably ten, adjacent nucleotides of the third nucleic acid portion, dependent on the number of nucleotides present in the third nucleic acid portion.

27. A conjugate according to claim 23, which comprises a phosphorothioate or phosphorodithioate internucleotide linkage between each adjacent nucleotide that is present in said third nucleic acid portion.

28. A conjugate according to claim 1, which comprises a phosphorothioate or phosphorodithioate internucleotide linkage linking the first nucleic acid portion to the third nucleic acid portion and/or the second nucleic acid portion to the third nucleic acid portion.

29. A conjugate according to claim 23, wherein at least one nucleotide of the first and/or second and/or third nucleic acid portion is modified.

30. A conjugate according to claim 29, wherein one or more of the odd numbered nucleotides starting from the 5′ region of the first nucleic acid portion are modified, and/or wherein one or more of the even numbered nucleotides starting from the 5′ region of the first nucleic acid portion are modified, wherein typically the modification of the even numbered nucleotides is a second modification that is different from the modification of odd numbered nucleotides.

31. A conjugate according to claim 29, wherein one or more of the odd numbered nucleotides starting from the 3′ region of the second nucleic acid portion are modified by a modification that is different from the modification of odd numbered nucleotides of the first nucleic acid portion according to claim 30.

32. A conjugate according to claim 29, wherein one or more of the even numbered nucleotides starting from the 3′ region of the second nucleic acid portion are modified by a modification that is different from the modification of odd numbered nucleotides of the second nucleic acid portion according to claim 31.

33. A conjugate according to claim 29, wherein at least one or more of the modified even numbered nucleotides of the first nucleic acid portion is adjacent to at least one or more of the differently modified odd numbered nucleotides of the first nucleic acid portion.

34. A conjugate according to claim 29, wherein at least one or more of the modified even numbered nucleotides of the second nucleic acid portion is adjacent to at least one or more of the differently modified odd numbered nucleotides of the second nucleic acid portion.

35. A conjugate according to claim 29, wherein a plurality of adjacent nucleotides of the first nucleic acid portion are modified by a common modification.

36. A conjugate according to claim 29, wherein a plurality of adjacent nucleotides of the second nucleic acid portion are modified by a common modification.

37. A conjugate according to claim 35, wherein said plurality of adjacent commonly modified nucleotides are 2 to 4 adjacent nucleotides, preferably 3 or 4 adjacent nucleotides.

38. A conjugate according to claim 37, wherein said plurality of adjacent commonly modified nucleotides are located in the 5′ region of the second nucleic acid portion.

39. A conjugate according to claim 37, wherein said plurality of adjacent commonly modified nucleotides are located in third nucleic acid region.

40. A conjugate according to claim 29, wherein a plurality of odd numbered nucleotides of the first and/or second nucleic acid portions are modified.

41. A conjugate according to claim 29, wherein a plurality of even numbered nucleotides of the first and/or second nucleic acid portions are modified by a second modification.

42. A conjugate according to claim 40, wherein said plurality of odd numbered nucleotides are modified by a common modification.

43. A conjugate according to claim 41, wherein said plurality of even numbered nucleotides are modified by a common second modification.

44. A conjugate according to claim 29, wherein the one or more of the modified nucleotides of the first nucleic acid portion do not have a common modification present in the corresponding nucleotide of the second nucleic acid portion of the duplex.

45. A conjugate according to claim 29, wherein the one or more of the modified nucleotides of the first nucleic acid portion do have a common modification present in the corresponding nucleotide of the second nucleic acid portion of the duplex.

46. A conjugate according to claim 29, wherein the one or more of the modified nucleotides of the first nucleic acid portion are shifted by at least one nucleotide relative to a commonly modified nucleotide of the second nucleic acid portion.

47. A conjugate according to claim 29, wherein the modification and/or modifications are each and individually sugar, backbone or base modifications, and are suitably selected from the group consisting of 3′-terminal deoxy-thymine, 2′-0-methyl, a 2′-deoxy-modification, a 2′-amino-modification, a 2′-alkyl-modification, a morpholino modification, a phosphoramidate modification, phosphorothioate or phosphorodithioate group modification, a 5′ phosphate or 5′ phosphate mimic modification and a cholesteryl derivative or a dodecanoic acid bisdecylamide group modification.

48. A conjugate according to claim 29, wherein the modification is any one of a locked nucleotide, an abasic nucleotide or a non-natural base comprising nucleotide.

49. A conjugate according to claim 29, wherein at least one modification is a 2′-O-methyl modification in a ribose moiety.

50. A conjugate according to claim 29, wherein at least one modification is a 2′-F modification in a ribose moiety.

51. A conjugate according to claim 29, wherein the nucleotides at any of positions 2 and 14 downstream from the first nucleotide of the 5′ region of the first nucleic acid portion do not contain 2′-O-methyl modifications in ribose moieties.

52. A conjugate according to claim 29, wherein the nucleotides of the second nucleic acid portion, that correspond in position to any of the nucleotides of the first nucleic acid portion at any of positions 9 to 11 downstream from the first nucleotide of the 5′ region of the first nucleic acid portion do not contain 2′-O-methyl modifications in ribose moieties.

53. A conjugate according to claim 51, wherein the nucleotides at any of positions 2 and 14 downstream from the first nucleotide of the 5′ region of the first nucleic acid portion contain 2′-F modifications in ribose moieties.

54. A conjugate according to claim 51, wherein the nucleotides of the second nucleic acid portion, that correspond in position to any of the nucleotides of the first nucleic acid portion at any of positions 9 to 11 downstream from the first nucleotide of the 5′ region of the first nucleic acid portion contain 2′-F modifications in ribose moieties.

55. A conjugate according to, claim 1 which comprises one or more unmodified nucleotides.

56. A conjugate according to claim 55, wherein said one or more unmodified nucleotides can replace any modified nucleotide as defined in any of claims 29 to 55.

57. A conjugate according to claim 56, wherein said one or more, preferably one, unmodified nucleotides represent any of the nucleotides at any of positions 17, 18, 19, 20, 21, 22, 23, 24 and/or 25 downstream from the first nucleotide of the 5′ region of the first nucleic acid portion, preferably positions 18, 19, 20 and/or 21.

58. A conjugate according to claim 51, wherein all nucleotides other than the unmodified nucleotides, and/or the nucleotides at any of positions 2 and 14 downstream from the first nucleotide of the 5′ region of the first nucleic acid portion, and/or the nucleotides of the second nucleic acid portion, that correspond in position to any of the nucleotides of the first nucleic acid portion at any of positions 9 to 11 downstream from the first nucleotide of the 5′ region of the first nucleic acid portion, contain 2′-O-methyl modifications in ribose moieties.

59. A conjugate according to claim 1, wherein the nucleic acid comprises at least one vinylphosphonate modification, such as at least one vinylphosphonate modification in the 5′ region of the first nucleic acid portion.

60. A conjugate according to claim 1, wherein one or more nucleotides of at least one of the first nucleic acid portion and the second nucleic acid portion is an inverted nucleotide and is attached to the adjacent nucleotide via the 3′ carbon of the nucleotide and the 3′ carbon of the adjacent nucleotide, and/or one or more nucleotides of at least one of the first nucleic acid portion and the second nucleic acid portion is an inverted nucleotide and is attached to the adjacent nucleotide via the 5′ carbon of the nucleotide and the 5′ carbon of the adjacent nucleotide.

61. A conjugate according to claim 60, wherein one or more nucleotides at the 3′ region of at least one of the first nucleic acid portion and the second nucleic acid portion is an inverted nucleotide and is attached to the adjacent nucleotide via the 3′ carbon of the terminal nucleotide and the 3′ carbon of the adjacent nucleotide, and/or one or more nucleotides at the 5′ region of at least one of the first nucleic acid portion and the second nucleic acid portion is an inverted nucleotide and is attached to the adjacent nucleotide via the 5′ carbon of the terminal nucleotide and the 5′ carbon of the adjacent nucleotide, and/or one or more nucleotides intermediate the 3′ and 5′ regions of at least one of the first nucleic acid portion and the second nucleic acid portion is an inverted nucleotide and is attached to the adjacent nucleotide via the 3′ carbon of the terminal nucleotide and the 3′ carbon of the adjacent nucleotide and/or one or more nucleotides intermediate the 3′ and 5′ regions of at least one of the first nucleic acid portion and the second nucleic acid portion is an inverted nucleotide and is attached to the adjacent nucleotide via the 5′ carbon of the terminal nucleotide and the 5′ carbon of the adjacent nucleotide, and/or one or more nucleotides of at least one of the third nucleic acid portion is an inverted nucleotide and is attached to the adjacent nucleotide via the 3′ carbon of the terminal nucleotide and the 3′ carbon of the adjacent nucleotide and/or one or more nucleotides of at least one of the third nucleic acid portion is an inverted nucleotide and is attached to the adjacent nucleotide via the 5′ carbon of the terminal nucleotide and the 5′ carbon of the adjacent nucleotide.

62. A conjugate according to claim 60, wherein the inverted nucleotide is attached to the adjacent nucleotide via a phosphate group by way of a phosphodiester linkage; or the 3′ and/or 5′ inverted nucleotide of the first and/or second strand is attached to the adjacent nucleotide via a phosphorothioate group; or the 3′ and/or 5′ inverted nucleotide of the first and/or second strand is attached to the adjacent nucleotide via a phosphorodithioate group.

63. A conjugate according to claim 1, which has blunt ended.

64. A conjugate according to claim 1, wherein either the first or second nucleic acid portion has an overhang.

65. A conjugate according to claim 1, which is a homo-dimer RNA molecule comprising two nucleic acid molecules as defined in claim 1, wherein said nucleic acid molecules are bound together through complementary interactions, where the first portion of the first molecule interacts with the second portion of the second molecule and there is a third portion in each molecule that generates a bulge structure intermediate of the first and second portions of the respective nucleic acid molecules.

66. A conjugate according to claim 1, wherein the target RNA is selected from at least one of: mRNA, IncRNA, and/or other RNA molecules.

67. A composition comprising a conjugate according to claim 1, and a physiologically acceptable excipient.

68. A conjugate or molecule according to claim 1, for use in the treatment of a disease or disorder.

69. Use of a conjugate according to claim 1, in the manufacture of a medicament for treating a disease or disorder.

70. A method of treating a disease or disorder comprising administration of a conjugate according to claim 1, to an individual in need of treatment.

71. A method according to claim 70, wherein the conjugate is administered subcutaneously or intravenously to the individual.

72-74. (canceled)
